(19) United States
(12) Patent Application Publication
(10) Pub. No.: US 2014/0127332 A1
(43) Pub. Date: May 8, 2014

(54) EXTRACTS

(71) Applicant: Catherine M. BITLER, Menlo Park, CA (US)

(72) Inventor: Catherine M. BITLER, Menlo Park, CA (US)

(21) Appl. No.: 13/672,624

(22) Filed: Nov. 8, 2012

(51) Int. Cl. A61K 36/9068; A61K 36/63; A61K 36/28

(52) U.S. Cl. CPC A61K 36/9068; A61K 36/28; A61K 36/63
USPC 424/756; 424/725; 424/764

(57) ABSTRACT

Novel compositions which are useful in the treatment of mammals for the reduction of inflammation, especially in the gut, and which comprise an aqueous extract of an agricultural product. Preferred compositions comprise an aqueous extract of olive, or an aqueous extract of ginger. The compositions can optionally contain an aqueous extract of chamomile and/or an aqueous extract of artichoke and/or an aqueous extract of cinnamon.

### CROSS-REFERENCE TO RELATED APPLICATION

This application claims priority from and the benefit of Provisional Application No. 61/628,995, filed Nov. 9, 2011, the entire disclosure of which is incorporated by reference herein for all purposes.

### FIELD OF THE INVENTION

This invention relates to extracts of fruits, herbs and vegetables which can be used in the treatment of mammals.

### BACKGROUND OF THE INVENTION

Inflammatory disorders in mammals, in particular inflammation of the gut, are common and serious. However, the available treatments suffer from important deficiencies.

Currently, three main classes of drugs, the proton pump inhibitors (omeprazole); the H2 antagonists (Mylanta); and the anti-cholinergic/anti-spasmodics; are prescribed for diseases associated with gut inflammation. However, there are well-documented problems associated with the more effective drugs.

For example omeprazole is contraindicated if the patient is taking a variety of other drugs, including antibiotics. Serious stomach conditions may occur while taking antibiotics with omeprazole, including cramping, bloating, diarrhea, and nausea or vomiting. Omeprazole may increase the risk of developing fractures of the hip, wrist and spine, especially in people who are 50 years of age and older. Extended use of omeprazole may result in convulsions, seizures, irregular heartbeat, tremors, muscle spasms, or weakness.

With anti-cholinergics, the side effects include decreased secretions, slowed gastrointestinal motility, blurred vision, increased heart rate, heat intolerance, sedation, and sometimes mild confusion. While the side effects in a younger person of good health are uncomfortable, in an elderly person the side effects could be disastrous. For example, increases in heart rate may precipitate or worsen angina; and, thermoregulatory impairment, which blocks the ability to sweat properly, may lead to life threatening hyperthermia.

Some natural compounds have anti-inflammatory activity, but their effectiveness is limited.

Therefore, there is a need for new treatments for controlling (i.e. preventing or reducing) inflammation, particularly inflammation of the gut.

### SUMMARY OF THE INVENTION

This invention provides novel compositions which are useful in the treatment of mammals for the reduction of inflammation, especially in the gut, and which comprise an aqueous extract of an agricultural product, e.g. a stone-bearing fruit (also known as a drupe) and/or a rhizome.

In a first preferred aspect, this invention provides compositions which are useful in the treatment of mammals for reduction of inflammation, especially in the gut, and which comprise an aqueous extract of olive. The compositions of the first aspect of the invention can optionally also contain one or more of an aqueous extract of ginger, an aqueous extract of chamomile, and an aqueous extract of artichoke.

In a second preferred aspect, this invention provides compositions which are useful in the treatment of mammals for the reduction of inflammation, especially in the gut, and which comprise an aqueous extract of ginger. Compositions of the second preferred aspect of the invention can optionally also contain one or more of an aqueous extract of olive, an aqueous extract of chamomile, and an aqueous extract of artichoke.

In a third preferred aspect, this invention provides novel compositions which are useful in the treatment of mammals for the reduction of inflammation, especially in the gut, and which comprise 6-shogaol and one or both of 6-gingerol and 8-shogaol. In compositions containing both 6-gingerol and 8-shogaol. the ratio of 6-gingerol to 6-shogaol is preferably 60:1 to 1:30, particularly 30:1 to 1:5. In compositions containing both 6-shogaol and 8-shogaol, the ratio of 6-shogaol to 8-shogaol is preferably 25:1 to 1:5, particularly 5:1 to 1:2. These compositions are preferably prepared by a process which comprises a step in which an aqueous extract of ginger is prepared, but the invention includes such compositions prepared in other ways.

Preferred compositions of the invention also have at least one of (i.e. one or more of) characteristics (A), (B) and (C) below:—
(A) the composition contains hydroxytyrosol and caffeic acid, the ratio of hydroxytyrosol to caffeic acid being 200:1 to 1:1, particularly 50:1 to 5:1, especially 25:1 to 10:1;
(B) the composition contains caffeoylquinic acids and apigenin glucuronide, the ratio of caffeoylquinic acids to apigenin glucuronide being 250:1 to 1:1, particularly 50:1 to 2:1, especially 25:1 to 4:1 and
(C) The composition contains apigenin and quercetin, the ratio of apigenin to quercetin being 100:1 to 1:20, particularly 30:1 to 1:5, especially 3:1 to 1:1.

Preferred ratios by weight of different ingredients in compositions of the invention, standardizing to 6-gingerol for ginger; hydroxytyrosol for olive; caffeoylquinic acids acid for artichoke; and apigenin for chamomile: are:—

Ginger/Olive—1:50

Ginger/Olive/Artichoke—1:50:2

Ginger/Olive/Chamomile—1:50:10

Ginger/Artichoke—1:2

Ginger/Chamomile—1:10

Olive/Artichoke—25:1

Olive/Chamomile—5:1

Artichoke/Chamomile—1:5

In a fourth preferred aspect, this invention provides methods of treating mammals which make use of a composition according to the first, second or third preferred aspect of the invention to control (i.e. to prevent or reduce) inflammation, particularly inflammation of the gut.

### BRIEF DESCRIPTION OF THE DRAWINGS

The invention is illustrated in the accompanying drawings, in which

FIGS. 1, 2 and 3 are graphs showing the results of in vitro experiments which demonstrate the effect of the compositions on lipoxygenase activity, and

FIGS. 4 and 5 are the bar graphs summarizing the results of using the compositions to treat inflammation in dogs.

### DETAILED DESCRIPTION OF THE INVENTION

In the Summary of the Invention above and in the Detailed Description of the Invention below, reference is made to particular features of the invention. It is to be understood that the disclosure of the invention in this specification includes all possible combinations of such particular features. For example, where a particular feature is disclosed in the context of a particular aspect or a particular embodiment, that feature can also be used in combination with other particular aspects and embodiments, and in the invention generally, except where the context excludes that possibility. The invention disclosed herein include embodiments not specifically described herein and can for example make use of features which are not specifically described herein, but which provide functions which are the same, equivalent or similar to, features specifically disclosed herein.

The term “comprises” and grammatical equivalents thereof are used herein to mean that, in addition to the features specifically identified, other features are optionally present. For example, a composition or device “comprising” (or “which comprises”) components A, B and C can contain only components A, B and C, or can contain not only components A, B and C but also one or more other components. The terms “consisting essentially of” and grammatical equivalents thereof are used herein to mean that, in addition to the features specifically identified, other features may be present which do not materially alter the claimed invention. The term “at least” followed by a number is used herein to denote the start of a range beginning with that number (which may be a range having an upper limit or no upper limit, depending on the variable being defined). The term “at most” followed by a number is used herein to denote the end of a range ending with that number (which may be a range having 1 or 0 as its lower limit, or a range having no lower limit, depending upon the variable being defined). When a range is given as “(a first number) to (a second number)” or “(a first number)-(a second number)”, this means a range whose lower limit is the first number and whose upper limit is the second number. The terms “plural”, “multiple”, “plurality” and “multiplicity” are used herein to denote two or more than two features.

Where reference is made herein to “a” or “an” feature, this includes the possibility that there are two or more such features (except where the context excludes that possibility). Where reference is made herein to two or more features, this includes the possibility that the two or more features are replaced by a lesser number or greater number of features providing the same function, except where the context excludes that possibility. The numbers given herein should be construed with the latitude appropriate to their context and expression; for example, each number is subject to variation which depends on the accuracy with which it can be measured by methods conventionally used by those skilled in the art.

This specification incorporates by reference all documents referred to herein and all documents filed concurrently with this specification or filed previously in connection with this application, including but not limited to such documents which are open to public inspection with this specification.

Parts, percentages and ratios given in this specification are by weight unless otherwise noted.

The “ginger” used in some aspects of this invention is preferably zingiber officinalis, including its varieties, e.g. red, white etc. The “olive” used in some aspects of this invention is preferably olea europaea. The “chamomile” used in some aspects of this invention is preferably matricaria recutita. The “artichoke” used in some aspects of this invention is preferably cynara scolymus.

The term “aqueous extract” is used herein to mean any product obtained by a process which comprises a step in which an initial material is treated with an aqueous fluid to extract ingredients therefrom. Examples of aqueous fluids are mixtures of water and ethanol, water containing salt without acid, and water containing acid without salt. Optionally, the extract is treated with acid, e.g. to lower its pH to the range 1.5 to 4; the acid may for example be citric acid, acetic acid or another organic acid. Generally, the product of the extraction step will be concentrated by one or more additional steps, for example filtration and residue removal, and/or freeze drying. A freeze-dried product can then be suspended in water.

The initial material which is used to prepare an aqueous extract of ginger (when an aqueous extract of ginger is part of the composition) can be the raw ginger rhizome, or another ginger product, e.g. ginger leaves, freeze-dried ginger, steam-distilled ginger, dried ginger, ginger pulp, ginger oil, ginger-derived water or ginger from supercritical extraction. The ginger may be organic ginger.

The initial material which is used to prepare an aqueous extract of olive (when an aqueous extract of olive is part of the composition) can be raw, de-pitted olives, or another olive product, e.g. olive leaves, olive extracts, olive tree stems and branches, freeze-dried olives or olive byproducts, steam-distilled olives, dried olives, olive pulp, olive oil, olive-derived vegetarian water and olive products from supercritical fluid extraction. The olive may be organic olive.

The initial material which is used to prepare an aqueous extract of artichoke (when an aqueous extract of artichoke is part of the composition) can be raw artichokes, or another artichoke product, e.g. artichoke leaves, artichoke extracts, artichoke stems and branches, freeze-dried artichoke, artichoke byproducts, steam-distilled artichoke, dried artichoke, artichoke pulp, artichoke oil, artichoke-derived vegetarian water. The artichoke may be organic artichoke.

The initial material which is used to prepare an aqueous extract of chamomile (when an aqueous extract of chamomile is part of the composition) can be chamomile leaves and/or chamomile flowers, or another chamomile product, e.g. chamomile extracts, chamomile stems and branches, freeze-dried chamomile, chamomile byproducts, steam-distilled chamomile, dried chamomile, chamomile pulp, chamomile or chamomile vegetarian water and chamomile products from supercritical fluid extraction. The chamomile may be organic chamomile.

The ingredients preferably extracted from ginger are at least two of 6-gingerol, 6-shogaol and 8-shogaol. The ingredients preferably extracted from olive are hydroxytyrasol and caffeic acid. The ingredients preferably extracted from artichoke are caffeoylquinic acid and apigenin glucuronide. The ingredients preferably extracted from chamomile are apigenin and quercetin.

Preferably, the olive extract (if used) comprises hydroxytyrosol and caffeic acid in a ratio of hydroxytyrosol to caffeic of 200:1 to 1:1, more preferably 50:1 to 5:1, most preferably a ratio of 25:1 to 10:1.

Preferably, the artichoke extract (if used) comprises caffeoylquinic acids and apigenin glucuronide in a ratio of caffeoylquinic acids to apigenin glucuronide of 250:1 to 1:1, more preferably 50:1 to 2:1, most preferably 25:1 to 4:1.

Preferably, the extract of chamomile (if used) comprises apigenin and quercetin in a ratio of apigenin to quercetin of 100:1 to 1:20, more preferably 30:1 to 1:5, most preferably a ratio of 3:1 to 1:1.

Specific examples of steps which can be taken in the aqueous extraction of ginger are as follows:—

(1) Raw ginger is sonicated or homogenized in a mixture of water and ethanol (30:70) and incubated for several hours. Following filtration and residue removal, the resultant extract is concentrated and freeze dried (lyophilized). The freeze dried powder is resuspended in water.

(2) Raw ginger is sonicated or homogenized in a mixture of water and ethanol (30:70) and incubated for several hours. Following filtration and residue removal, the resultant extract is treated with acid and/or salt, and is then freeze dried. The freeze dried powder is resuspended in water.

(3) Raw ginger is extracted with water or steam or both, and the product is the freeze-dried to produce a dried powder extract.

(4) Raw ginger is cleaned and then freeze-dried. The freeze-dried product is subject to grinding and sieving, and the product is subjected to aqueous extraction, e.g. by sonication with ethanol and aqueous acid and then dried, ground and sieved.

Specific examples of steps that can be taken in the aqueous extraction of olive are as follows.
(1) Raw olives are washed, depitted, and macerated in water containing salt and acid with heat for several hours. The olive extract is centrifuged by 3-phase centrifugation, yielding olive juice, olive oil, and olive pulp (particulate). The olive juice is incubated, e.g. for several months, and then subjected to filtration and/or pasteurization.
(2) Raw olives are macerated in water containing salt without acid, with heat.
(3) Raw olives are macerated in water containing acid without salt, with heat.
(4) The treated olive juice is freeze dried (lyophilized) to a powder.

Artichoke Extract

Specific examples of steps which can be taken in the aqueous extraction of artichoke are as follows:
(1) Raw artichokes are extracted with a mixture of water and ethanol (e.g. 50:50) under heat. The resultant extract is freeze-dried to a powder.
(2) Raw artichokes are extracted with a mixture of water and ethanol (e.g. 50:50) and acid and/or salt, and then freeze dried to a powder.

Specific examples of steps which can be taken in the aqueous extraction of chamomile are as follows:
(1) Dry chamomile flowers are crushed and subjected to boiling water, followed by shaking (e.g. at 200 rpm and 37° C.) for 4-6 hours.
(2) Chamomile flowers are crushed and subjected to boiling water, followed by shaking (e.g. at 200 rpm and 37° C.) for 4-6 hours, after which the extract is brought to 5% ethanol, and shaking (e.g. at 200 rpm and 37° C.) is continued at for an additional 4-6 hours.

When administering compositions of the invention which contain a ginger extract, for example in the form of a pharmaceutical composition, the amount of ginger is preferably at least 0.5 mg/kg body weight/day, preferably 1-500 mg/kg body weight/day, particularly 2-250 mg/kg body weight/day. The dosage can be in the form of a single dose or multiple doses.

The compositions of the invention can optionally contain, as an additional ingredient, an aqueous extract of cinnamon

The remainder of the detailed description of the invention provides further information about preferred embodiments of the invention.

In some preferred embodiments of the invention, a composition comprising an aqueous fraction of ginger in combination with one or more of the following: olive (olea europaea) extract, chamomile (matricaria recutita) extract, or artichoke (cynara scolymus) extract, is used to promote gut health and to decrease or prevent inflammation and/or disease of the gut associated with, for example, cancer, autoimmune disease, physical stress, diet, and/or aging.

In some preferred embodiments of the invention, an aqueous fraction of the ginger plant acts with the other natural plant extracts, specifically one or more of olive, chamomile and artichoke, for example to decrease or prevent inflammation of the gut associated with, for example, physical stress, diet, aging or disease, to promote gut and digestive health, and to relieve the gut of pain and/or nausea associated with stress, diet, aging and disease.

In some preferred embodiments of the invention, the novel compositions are used to treat humans. In other preferred embodiments, the novel compositions are used to treat animals, particularly animals that are under physical stress (e.g. sled dogs, sporting dogs, K9s, working horses, polo ponies, eventing horses, steeplechasers and racehorses), animals that suffer stress associated with travel (automobile, train, airplane etc.), animals that are older, and animals that have been diagnosed with a disease that results in local or systemic inflammation.

It is believed (though the invention is not dependent upon the truth of this belief) that the novel compositions of the invention protect and promote gut health in at least two ways that are not directly associated with antimicrobial activities.

First, it is believed that the compositions inhibit, in some cases through a synergistic combination of ingredients, the enzyme lipoxygenase. Increase in lipoxygenase activity results in an increase in the pro-inflammatory mediators leukotrienes. Inflammation in the gut is associated with acute conditions such as gastritis, nausea, and pain; and chronic conditions such as ulcers and cancer. Lipoxygenase is a non-heme iron-containing dioxygenase that catalyzes the stereo-specific peroxidation of polyunsaturated fatty acids containing at least one 1-cis, 4-cis-petadiene, such as linoleic, α-linolenic or arachadonic acids. Activation of lipoxygenases occurs during stress and disease results in the liberation of the pro-inflammatory leukotrienes. Leukotrienes activate NFkB, the transcriptional activator of numerous pro-inflammatory molecules, including cytokines, chemokines etc. Activation of this cascade increases local and or systemic inflammation resulting in pain, nausea, and following chronic inflammation, disease of the gut. The novel compositions inhibit the activity of the lipoxygenases thereby inhibiting the consequential inflammatory cascade.

Second, it is believed that the compositions decrease, in some cases synergistically, levels of TNF-α. A potent pro-inflammatory cytokine, TNF-α has multiple effects on a variety of cell types and plays an important role in the pathogenesis of chronic inflammation.

Third, it is believed that the compositions act as an antioxidant of free radicals. Reactive oxygen species, or free radicals, are a normal by-product of metabolism. However, under conditions of physical or chemical stress, aging or chronic illness, reactive oxygen species are particularly damaging to tissues, cells, proteins, and DNA.

Some embodiments of the invention relate to a composition of aqueous/alcohol ginger extract in combination with one or more of the following extracts: olive (olea europaea) extract, chamomile (matricaria recutita) extract, or artichoke (cynara scolymus). Preferred compositions contain an aqueous/alcohol ginger extract with a composition of 6-gingerol to 6-shogaol with a ratio of 60:1 to 1:30, more preferably a ratio of 30:1 to 1:5; and a composition of 6-shogaol to 8-shogaol with a ratio of 25:1 to 1:5, more preferably a ratio of 5:1 to 1:2.

It has surprisingly been found that in some cases the combination of an aqueous ginger extract and one or more of an aqueous extract of olive, an aqueous extract of chamomile, and an aqueous extract of artichoke, functions synergistically in their anti-inflammatory activity, which makes them particularly useful for in the treatment, co-treatment, and prevention of inflammatory disorders, especially inflammatory disorders of the gut.

The compositions of the present invention are particularly suitable for the treatment, co-treatment and prevention of different inflammatory gut disorders, in particular gastritis and sickness from disequilibrium. The compositions of the present invention are suitable for treatment, co-treatment and prevention of gut disorders in particular reduction of inflammation, inhibition of inflammatory mediators, maintenance and/or increase of gut health, prevention of gut nausea, decrease in free radicals, increase in ratio of beneficial microbes to deleterious microbes, providing an environment for maintenance of beneficial microbes, decrease in pain, prevention of ulcers, and prevention of cancerous cell growth.

The invention also relates to the use of the compositions of the invention as an agent for the treatment, co-treatment or prevention of inflammatory disorders and disorders of aging. Inflammatory disorders include arthritis (osteoarthritis and rheumatoid arthritis), asthma, inflammatory bowel disorder, and inflammatory diseases of the skin (e.g. contact dermatitis, atopic dermatitis, eczema, psoriasis, rosacea, seborrhea, burns, and other types of skin inflammation associated with aging), chronic inflammatory disorders including heart disease, atherosclerosis, Parkinson's disease, Alzheimer's disease, autoimmune disorders, cancer, and diseases associated with epigenetic modulation.

The invention also relates to use of the compositions of the invention as a medicament.

In one embodiment, the composition according to the invention is incorporated into a nutraceutical, dietary supplement, nutritional supplement, pharmaceutical, functional food, food, or prophylactic suitable for the treatment, co-treatment, or prevention of inflammatory disorders, more preferably of gut or digestive tract inflammation, most preferably for gastritis or nausea.

The invention includes methods for the treatment, co-treatment and prevention of inflammatory disorders, in particular gut inflammation, more in particular gastritis or disequilibrium sickness, in animals, including humans, said method comprising the step of administering an effective amount of the composition according to the invention to animals including humans, which are in need thereof.

The term “an effective amount of the composition” means an amount which exerts a useful physiological effect. The dosage administered may vary depending upon known factors such as the physiological characteristics of the composition and its mode and route of administration, the age, health and weight of the recipient, the nature and extent of symptoms, the frequency of treatment and the desired effect etc.

The term “animals” means all animals, including mammals, which includes humans. Preferred examples of mammals beside humans are ruminant and non-ruminant animals including dogs, cats, horses, cows, sheep, goats, and elephants.

The term “nutraceutical” includes food products, foodstuff, dietary supplement, nutritional supplement, or a supplement for food product or foodstuff.

The term “food product” means any food or feed suitable for consumption by humans or animals. The food product may for example be a prepared and packaged food or an animal feed. The term “foodstuff” means any substance fit for human or animal consumption. The term “dietary supplement” means a composition which is used in a small amount for supplementation of a human or animal diet and which is packaged in single or multiple dose units. Dietary supplements do not generally provide significant amounts of calories but may contain other micronutrients (e.g. vitamins, minerals). The term “nutritional supplement” means a composition comprising a dietary supplement in combination with a source of calories. In some embodiments, nutritional supplements are functional foods or meal replacements.

Food products (or foodstuffs) include for example liquid preparations to be added to drinking water, solid food or liquid food, and sport drinks, fruit juices, carbonated drinks, teas, combination water drinks, milk, other dairy drinks and weight control drinks. In another embodiment food products may refer to a solid or semi-solid food comprising the composition of the invention. Such products can include but are not limited to baked products such as biscuits, treats, snack foods, meat products, vegetarian products, dairy products, cookies, cakes, confections, frozen meals, raw food diets, liquid foods including soups, sauces, spreads, dressings. Other products may include cheeses, yogurts, foodstuffs containing an oil or fat, and food ingredients such as flour, rice, millet etc. The term food and foodstuffs includes functional foods and prepared food products.

Animal feed and pet food compositions include food intended to supply necessary dietary requirements, as well as treats or other food supplements. An animal feed comprising a composition according to the invention may be in the form of a dry composition, semi-moist composition, wet composition, or any mixture thereof. Alternatively or additionally, the animal feed is a supplement such as in the form of gravy, water, yogurt, powder, suspension, chew, treat or any other delivery form.

Dietary supplements may be delivered in any suitable format. In the preferred embodiments, dietary supplements are formulated for oral delivery. The ingredients of the dietary supplement are combined with acceptable excipients and/or carriers for oral consumption. The carrier may be a liquid, emulsion, gel, capsule, powder, solid tablet, solid or liquid food, tea, or the like. The dietary supplement is preferably in the form of a liquid, capsule or tablet. Suitable excipient and/or carriers include maltodextrin, celluloses, vegetable gums, glycerin, cornstarch, flour, sodium, sucrose, lactose, calcium carbonate, calcium phosphate, stearic acid, magnesium stearate, citric acid, and the like, including mixtures thereof. The various ingredients and the excipient and/or carrier are mixed and formed into the desired form using conventional techniques. The tablet or capsule may be uncoated or coated with an enteric coating that dissolves at a pH greater than 6.

In other embodiments, the dietary supplement is a liquid or powder suitable for adding to a food or beverage. For example, in some embodiments, the dietary supplement can be administered to an individual in the form of a powder, for example to be used by mixing into a liquid, a semi-solid food or adding to a food. The dietary supplement may be encapsulated or contained within another food grade substance to be released immediately prior to consumption. The dietary supplement may be composed of one or more inert ingredients. For example, the dietary supplement may contain colorants, flavorants, sweeteners, and the like. The dietary supplement may also contain optional ingredients including, for example herbs, spices, vitamins and minerals.

In some embodiments, the dietary supplements further comprise vitamins and minerals, including, but not limited to, calcium phosphate or acetate, potassium phosphate, magnesium sulfate, sodium chloride, potassium chloride, ascorbic acid, iron, niacinamide, nicotinamide riboside, calcium pantothenate, zinc sulfate or oxide, copper gluconate, riboflavin, beta-carotene, thiamin, pyridoxine hydrochloride, folic acid, biotin, chromium chloride or piconolate, potassium iodide, sodium selenate, sodium selenite, sodium molybdate, vitamin D3, copper sulfate, vitamin A, vitamin C, inositiol. Suitable dosages for vitamins and minerals may be obtained, for example, by consulting the U.S. RDA guidelines.

In some embodiments, the present invention provides nutritional supplements comprising a composition according to the invention. Nutritional supplements may serve as a meal replacement or snack and generally provide calories for nutrition. Preferably, the nutritional supplements provide carbohydrates, proteins, and fats in balanced amounts.

The nutritional supplement can optionally further comprise a simple or medium length carbohydrate, or a polysaccharide, or combinations thereof. If it is desired that the carbohydrate should maintain its high molecular weight structure, it should be included only in food formulations which are not cooked or heat processed as heat will be breakdown the complex carbohydrate into simple carbohydrates, including mono- or di-saccharides. The nutritional supplement may contain protein. The sources of protein incorporated into the nutritional supplement can be any suitable protein utilized in nutritional formulations and can include fish protein, chicken protein, egg protein, whey protein, soy protein, soy milk protein, caseinate, animal and vegetable protein and hydrolysates or mixtures thereof. When choosing a protein source, the biological value of the protein should be considered first, with the highest biological values being found in caseinate, whey, lactalbumin, egg albumin and whole egg proteins. In a preferred embodiment the protein is a combination of whole egg proteins and fish protein.

A nutritional supplement can also optionally contain other ingredients such as one or a combination of other vitamins, minerals, antioxidants, fiber and other dietary supplements. Selection of one or several of these ingredients is a matter of formulation, design, consumer preference and end-user. Further vitamins and minerals that can be added include, but are not limited to, calcium phosphate or acetate, potassium phosphate, magnesium sulfate, sodium chloride, potassium chloride, ascorbic acid, iron, niacinamide, nicotinamide riboside, calcium pantothenate, zinc sulfate or oxide, copper gluconate, riboflavin, beta-carotene, thiamin, pyridoxine hydrochloride, folic acid, biotin, chromium chloride or piconolate, potassium iodide, sodium selenate, sodium selenite, sodium molybdate, vitamin D3, copper sulfate, vitamin A, vitamin C, and inositiol.

Additionally, flavors, coloring agents, spices, nuts and the like may optionally be incorporated into a nutritional supplement composition. Flavorings can be in the form of flavored extracts, volatile oils, sweet flavorings including but not limited to chocolate flavorings, peanut butter flavoring, vanilla flavoring; and savory flavorings including but not limited to fish flavoring, butter flavoring, olive flavoring or any commercially available flavoring. Examples of useful volatile oils include walnut oil, fish oil, rosmarinic oil, turmeric oil, clove oil, cherry oil, cinnamon oil, and peppermint oil.

Emulsifiers may optionally with be added for stability of the nutritional supplement compositions. Examples of suitable emulsifiers include, but are not limited to, lecithin (e.g., from egg or soy), and/or mono- and di-glycerides. Preservatives may also be added to the nutritional supplement to extend product shelf life. Preferably, preservatives such as potassium sorbate, sodium sorbate, potassium benzoate, sodium benzoate or calcium disodium EDTA are used. In addition to the carbohydrates described above, the nutritional supplement composition can contain natural or artificial sweeteners, e.g., saccharides, cyclamates, aspartamine, aspartame, sorbitol and/or stevia (e.g. stevia rebaudiana, stevioside). Such artificial sweeteners can be desirable if the nutritional supplement is intended to be consumed by an overweight or obese individual, or an individual with type II diabetes who is prone to hyperglycemia.

In some embodiments, a multi-vitamin and mineral supplement may optionally be added to the nutritional supplement compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

In another aspect the invention relates to a pharmaceutical comprising the composition according to the invention and a pharmaceutically acceptable carrier.

A person skilled in the art will know, having regard to his own knowledge and the disclosure in this specification, which carriers can be used as pharmaceutically acceptable carriers. Examples of pharmaceutically acceptable carriers are both inorganic and organic carrier materials, suitable for delivery via oral, parenteral, intravenous, intramuscular, subcutaneous, sub-dermal or dermal routes.

The pharmaceutical composition may optionally further comprise conventional pharmaceutical additives and adjuvants, excipients or diluents.

In some embodiments, the pharmaceutical is in the form of a liquid, powder, tablet, capsule, gel, or solid.

A person skilled in the art will know, having regard to his own knowledge and the disclosure in this specification, how to select suitable dosages and ratios of the individual components in the pharmaceutical composition.

In a preferred embodiment, a composition of the second aspect of the invention is administered via a pharmaceutical composition either in the form of a single dose or multiple doses which contain ginger extract in an amount of at least 0.5 mg/kg body weight/day, preferably in an amount 1-500 mg/kg body weight/day, most preferably in an amount of 2-250 mg/kg body weight/day.

A nutraceutical or pharmaceutical according to the present invention may be in any form that is suitable for administering to the animal including humans, particularly in any form that is conventional for oral administration, e.g. in solid form, for example as supplements/additives as in food or feed, fortified food or feed, tablets, pills, granules, capsules and effervescent formulations such as powders and tablets, or in liquid form, for instance in the form of solutions, emulsions or suspensions, for example as beverages, pastes and oily suspensions. The pastes may be filled into hard or soft shell capsules. Examples for other application forms are forms for transdermal, parenteral, topical or injectable administration. The nutraceutical or pharmaceutical may be in the form of controlled (delayed) release formulations

### EXAMPLES

The olive extract used in some of the Examples contained hydroxytyrosol in amount about 2 milligram/ml and caffeic acid in amount about 20 microgram/ml. The ginger/olive extract used in some of the Examples contained 6-gingerol in amount about 45 microgram/ml, hydroxytyrosol in amount about 2 milligram/ml and caffeic acid in amount about 20 microgram/ml.

### Example 1

### Inhibition of Inflammation Mediators by Ginger and Olive Extracts

Lipoxygenase catalyzes the conversion of arachadonic acid to leukotrienes, which are mediators of local inflammation. Lipoxygenase activation increases during periods of physical or chemical stress and chronic activation results in long term or chronic states of dysfunction and/or disease. A substance that inhibits lipoxygenase, especially during periods of stress, favors a state of decreased inflammation and a lower probability of disability from chronic dysfunction or disease.

In vitro experiments were carried out to determine the effect on lipoxygenase activity of the aqueous olive extract alone, the aqueous ginger extract alone, and a combination of the aqueous olive extract and the aqueous ginger extract. Lipoxygenase and its substrates (arachadonic acid and linoleic acid) were purchased from Cayman Chemical. A control experiment with no extracts was also carried out. The extract or mixture of extracts was incubated with the enzyme plus substrate for 10 minutes, after which the reaction was stopped with a chromagenic substrate and developed for 5 minutes.

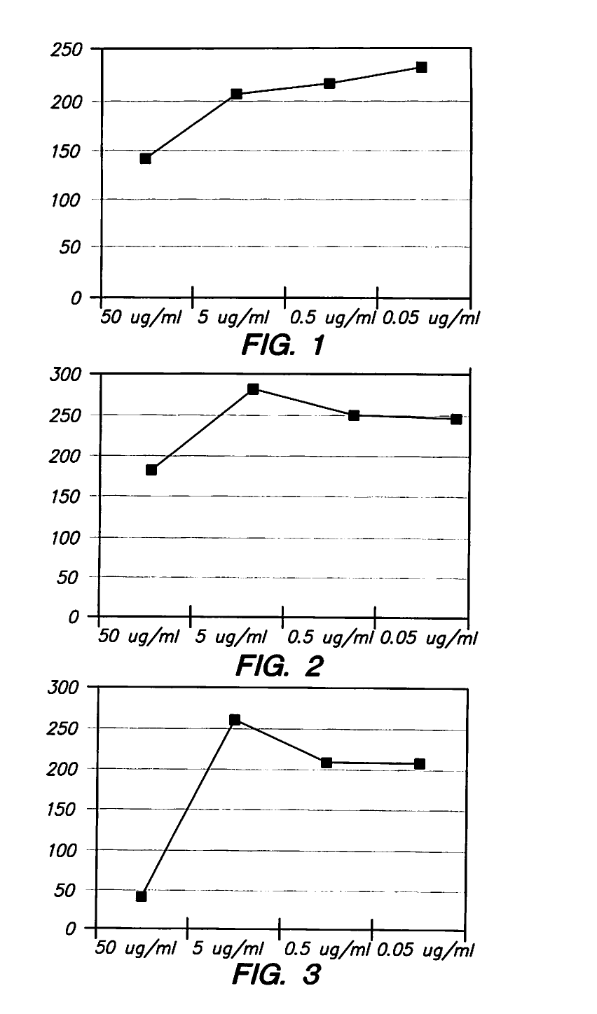

A standardized (6-shogaol) aqueous ginger extract was used at concentrations in the range of 1-100 micro molar 6-shogaol. A standardized aqueous olive extract (HT) was used at concentrations in the range of 1-100 micro molar. A combination of the aqueous olive extract and aqueous ginger extract was prepared by mixing equal quantities of the extracts at a temperature of 80-85° C. for 5 minutes. The mixture was cooled before being incubated with the enzyme plus substrate. The mixture was also used in the concentration range of 1-100 micro molar. The results are shown in FIGS. 1, 2 and 3, with the concentration of the extract or extracts on the x-axis, and the extent that lipoxygenase was inhibited on the y-axis, as shown by a decrease in color measured at wavelength 490 nm. FIG. 3 shows that when the two extracts are combined, a synergistic result is obtained.

### Example 2

### Decreased Pain and Improved Mobility in Dogs with Osteoarthritis Following An Eight-Week Treatment with Olive Extract

A double blind, placebo-controlled, randomized clinical trial lasting 8 weeks with 40 dogs was run to evaluate the effects of a placebo and the olive extract in older dogs with veterinarian-diagnosed osteoarthritis. Both the owner and the veterinarian evaluated the health of the dogs at the beginning and at the end of the eight-week trial.

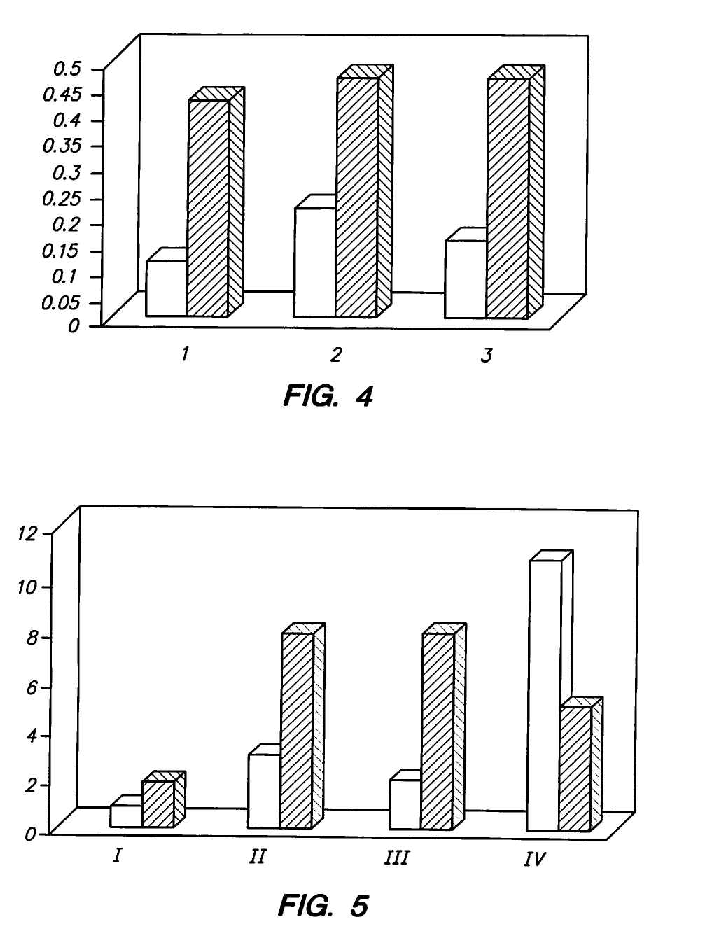

Results of the trial are shown in FIGS. 4 and 5, in which the lighter color vertical bars relates to the placebo, and the darker color vertical bars relate to the olive extract. FIG. 4 shows the assessment by the veterinarian of (1) pain (p=0.02), (2) ambulation (p=0.04), and (3) synovial fluid effusion (p=0.08). As seen in FIG. 4, the dogs treated with the aqueous olive extract showed improvement in the veterinarian's assessments of pain, mobility (ambulation) and synovial fluid effusion, with statistically significant improvement for mobility and pain.

FIG. 5 shows veterinarian's grading of the dogs' osteoarthritis. The y-axis shows the number of dogs in each of four categories, and the x-axis shows the four categories. In category II, the response to the treatment was assessed as 60-85% positive. In category III, the response to the treatment was assessed as 30-60% positive. In category IV, the response to the treatment was assessed as no response. FIG. 5 shows a statistically significant difference in improvement between the placebo and treatment groups, with 10 dogs showing at least 60% improvement (vs. 4 dogs in placebo group), and 12 dogs in the placebo group showing no response (vs. 6 dogs in the treatment group).

### Example 3

### Elimination of Motion Sickness in Young Dogs Following Treatment with Aqueous Ginger Extract Combined with Aqueous Olive Extract

A litter (4) of Borzoi puppies with extreme motion sickness were pre-treated with an aqueous ginger/olive extract before a multi day car trip. Prior to treatment with the aqueous ginger/olive extract, all 4 puppies suffered from hyper-emesis when traveling by car, even on very short car trips. Treatment with the aqueous ginger/olive extract included addition of the extract to their food 24 hr prior to travel and then twice a day during the trip. After the trip, the puppies were administered the aqueous ginger/olive extract twice a day for several more weeks.

Following the initial 24 hr treatment, the puppies showed no signs of gastric discomfort nor did any of the puppies vomit on the trip. It was noted that both during and after the trip the puppies appeared less agitated possibly due to the decreased gastric upset.

### Example 4

### Treatment of Puppies and Young Dogs with Aqueous Ginger/Olive Extract Improves Joint and Muscle Health

In an open clinical trial, a litter of Labrador Retriever puppies were either given a dose of olive/ginger extract or not twice a day with normal feeding following an 8 week weaning period. At 12 weeks, the puppies began a typical training period for sporting/hunting dogs. The puppies in the treatment group showed better muscle coordination, increased duration, better appetite, and fewer joint injuries than puppies in the non-treated group.

### Example 5

### Treatment of Extreme Athlete Dogs with Aqueous Olive Extract Decreases Stress-Related Gastric Upset, Improves Appetite, and Decreases Joint and Muscle Related Injuries

Sled dog teams from 3 different kennels were treated with the aqueous olive extract during training, during racing, and immediately following the racing season. The training/racing seasons included 2008, 2009, 2010, and 2011 seasons. Races included Iditarod, John Beargrease etc.

Sled dogs are extreme athletes that are expected to perform under the most difficult conditions. They train for several months prior to the racing season and then race under the harshest, most demanding conditions. While joint and muscle problems are common, one of the most critical conditions that afflict these dogs are gastric upset and sometimes ulcers that impair their ability to eat which prevents them from further performing. To assess the efficacy of aqueous olive extract on extreme athlete dogs, six sled dog teams from 3 different kennels were treated with the aqueous olive extract in an open trial.

Kennel 1—Dogs were treated with aqueous olive extract for 12 weeks prior to racing season and then during each of the races. As reported by the dogs' trainer/musher, the dogs were healthier; less fatigued, and had improved appetites over the same or similar dogs during previous race seasons. Additionally, there were no reported injuries to the dogs nor was there any incidence of gastric upset, including nausea, vomiting, refusal to eat and/or ulcers. The supplement-treated dogs in this kennel placed first in the longest sled dog marathon race in the lower 48 states (the John Beargrease sled dog marathon). In addition, these dogs received the “Best Kept Dog Award.” The “Best Kept Dog Award” is awarded to the healthiest team of dogs as assessed by a panel of 14 veterinarians. The assessments are performed prior to, during, and following the race. The dogs from this kennel had never before received this designation.

Kennel 2—Dogs were treated with the aqueous olive extract for several weeks prior to racing season and then during each of the races. As reported by the dogs' trainer/musher, the dogs were healthier and less fatigued than in previous race seasons. Additionally, there were no reported injuries nor was there any incidence of gastric upset, including nausea, vomiting, refusal to eat and/or ulcers. It was reported that the dogs had more energy during each of the races, and presumably as a result of the improved health and greater energy, the dogs finished in superior position than previous year races.

Kennel 3—A high performing dog sled team was treated with the aqueous olive extract for weeks prior to racing season and then during some of the races. As reported by the dogs' trainers and musher, the dogs responded well to treatment. The dogs were healthier, had fewer injuries and improvement in gastrointestinal health. The team consistently placed 1^{st} and or 2^{nd} in most races.

1-12. (canceled)

13. A method of treating a mammal for the reduction of inflammation in the gut, the method comprising administering to the mammal a composition which comprises at least one of (1) an aqueous extract of a stone-bearing fruit, (2) an aqueous extract of a rhizome, (3) a liquid or solid obtained by the treatment of an aqueous extract of a stone-bearing fruit, and (4) a liquid or solid obtained by the treatment of an aqueous extract of a rhizome.

14. (canceled)

15. A method according to claim 13 wherein the composition which is administered to the mammal further comprises one or more of an aqueous extract of ginger, an aqueous extract of chamomile, an aqueous extract of artichoke, and a liquid or solid obtained by the treatment of one or more of an aqueous extract of ginger, an aqueous extract of chamomile, and an aqueous extract of artichoke.

16. A method according to claim 13 wherein the composition which is administered to the mammal comprises an aqueous extract of ginger or a liquid or solid obtained by treatment of an aqueous extract of ginger.

17. A method according to claim 16 wherein the composition is an aqueous/alcohol ginger extract which contains 6-gingerol and 6-shogaol in a ratio of 30:1 to 1:5.

18. A method according to claim 16 wherein the composition contains 6-shogaol and 8-shogaol in a ratio of 5:1 to 1:2.

19. A method according to claim 16 wherein the composition contains 6-gingerol and 8-shogaol in a ratio of 5:1 to 1:2.

20. A method according to claim 13 wherein the composition comprises an aqueous extract of olive or a liquid or solid obtained by the treatment of an aqueous extract of olive.

21. A method according to claim 20 wherein the composition contains hydroxytyrosol and caffeic acid, the ratio of hydroxytyrosol to caffeic acid being 50:1 to 5:1.

22. A method according to claim 21 wherein the ratio of hydroxytyrosol to caffeic acid is 25:1 to 10:1.

23. A method according to claim 13 wherein the composition comprises an aqueous extract of artichoke which contains caffeoylquinic acids and apigenin glucuronide, the ratio of caffeoylquinic acids to apigenin glucuronide being 50:1 to 2:1.

24. A method according to claim 23 wherein the ratio of caffeoylquinic acids to apigenin glucuronide is 25:1 to 4:1.

25. A method according to claim 13 wherein the composition comprises an aqueous extract of chamomile or a liquid or solid obtained by the treatment of an aqueous extract of chamomile.

26. A method according to claim 25 wherein the composition comprises apigenin and quercetin, the ratio of apigenin to quercetin being 30:1 to 1:5.

27. A method according to claim 26 wherein the ratio of apigenin to quercetin is 3:1 to 1:1.

28. A method according to claim 13 wherein the mammal is a dog.

29. A method according to claim 28 wherein the composition is administered in a dosage of 1-500 mg per kilogram body weight per day.

30. A method according to claim 29 wherein the dosage is 2-250 mg per kilogram body weight per day.

31. A method according to claim 29 wherein the composition comprises an aqueous extract of olive or a liquid or solid obtained by the treatment of an aqueous extract of olive.

32. A method according to claim 31 wherein the composition contains hydroxytyrosol and caffeic acid, the ratio of hydroxytyrosol to caffeic acid being 50:1 to 5:1.

33. A method according to claim 32 wherein the ratio of hydroxytyrosol to caffeic acid is 25:1 to 10:1.
